(19) United States
(12) Patent Application Publication
(10) Pub. No.: US 2026/0304018 A1
(43) Pub. Date: Oct. 1, 2026

(54) SAMAEATI-HAJJ-SPECIAL NEEDS

(71) Applicant: Hamed Saigh BUDOR, Makkah Al Mukarramah (SA)

(72) Inventor: Hamed Saigh BUDOR, Makkah Al Mukarramah (SA)

(21) Appl. No.: 19/479,655

(22) PCT Filed: Mar. 12, 2024
(86) PCT No.: PCT/SA2024/000003

§ 371 Date: Oct. 28, 2025

(30) Foreign Application Priority Data
Jan. 20, 2024 (SA) SA 1020240303

(51) Int. Cl. H04R 1/10; A61B 5/00; G06F 40/58; H04R 1/08; H04R 1/1025

(52) U.S. Cl. CPC H04R 1/105; A61B 5/6822; G06F 40/58; H04R 1/08; H04R 1/1016; H04R 1/1025

(57) ABSTRACT

“Samaeati” (for Hajj and Umrah pilgrims, tourists, and businesspeople) is a neck-worn headset that provides instant real-time translation without the need to connect to a smartphone, using AI for all languages. It includes a GPS system to track the numbers of pilgrims and ensure that everyone is in their authorized area during rituals or within the service provider's region. The device is lightweight and comfortable for extended use, made from sweat-resistant, eco-friendly, and recyclable materials. It can measure temperature, pressure, and heart rate to protect against heatstroke. Solar panels are included for battery recharging without the need for a wall outlet during rituals. There's a directed speaker for ear-level sound delivery even when not worn inside the ear for emergency situations. It also features reverse translation through an integrated mic in the expandable and magnetically secured body. The inner earphone is expandable and can be secured using magnets.

### BACKGROUND ART

Pilgrims and Umrah performers face significant challenges while performing Islamic rituals due to the large and increasing number of attendees in Mecca and the holy sites. In 1444 Hijri, the number of pilgrims reached 1,845,045, with 1,660,915 coming from outside the Kingdom, according to the General Authority for Statistics. This number is expected to reach 3,000,000. The dominant age group among these pilgrims is over 65 years, who often lack skills in using smartphones and modern technology. Additionally, organizers and operating companies face challenges due to language barriers, as 80% of the pilgrims do not speak Arabic, as reported by Al Hurra newspaper in 1439 Hijri. In 1443 Hijri, the number of Umrah performers was 24,715,307 million, according to the General Authority for Statistics. According to the Ministry of Tourism in 1445 Hijri, the total number of tourists visiting the Kingdom for all purposes was 53.6 million.

There is a need for headphones that operate on an AI system without requiring connection to a smartphone. These headphones aim to provide instant translation for all languages and serve all target groups (pilgrims, Umrah performers, tourists, and businessmen), including those with special needs. The headphones will address several issues, including: 1) overcoming foreign language barriers; 2) managing crowds and congestion; 3) preventing the loss of pilgrims and Umrah performers; 4) addressing challenges faced by organizers and guides; 5) enabling early intervention in emergencies; 6) speeding up assistance delivery; 7) improving navigation and movement in cases of getting lost; and 8) reducing delays in reaching destinations.

### SUMMARY OF INVENTION

Samaeati (for pilgrims, umrah performers, tourists, and businessmen) is a neck-placed headset (FIG. 2) that provides instant, real-time translation without the need to connect to a smartphone, using AI for all languages (FIG. 12). It features a GPS system (FIG. 10) to track the number of pilgrims and ensure everyone is in their authorized area during rituals or within the service provider's area. The headset is lightweight and comfortable for extended use (FIG. 2), made from non-sweat-inducing, environmentally friendly, and recyclable materials (FIG. 10). It can measure temperature, pressure, and heart rate to protect against heatstroke due to prolonged sun exposure (FIG. 14). It includes solar panels for battery recharging without the need for wall sockets during the rituals (FIG. 13). The headset has a speaker directed towards the ear to deliver sound even when not placed inside the ear for emergency situations (FIG. 10). It also features a reverse translation capability through a built-in microphone in the body of the headset, which is extendable and fixable by a magnet (FIG. 10). The inner earpiece is also extendable and fixable by a magnet (FIG. 9).

### BRIEF DESCRIPTION OF DRAWINGS:

FIG. 1: Represents the overview of the headset.

FIG. 2: Illustrates the front view of the headset when worn.

FIG. 3: Shows the side view of the headset when worn.

FIG. 4: Depicts the rear view of the headset when worn.

FIG. 5: Displays the right part of the headset with the primary control buttons.

FIG. 6: Represents a close-up of the side part to highlight some components.

FIG. 7: Represents a close-up of the back part to illustrate some components.

FIG. 8: Shows the left part of the headset.

FIG. 9: Represents the headset when extended and removed from its magnetic holder.

FIG. 10: Depicts the top view of the headset, including:
10.1: Special rubber material.
10.2: Ear-directed speaker.
10.3: Extendable microphone fixed by a magnet.
10.4: GPS system.
10.5: Extendable inner earpiece (left).

FIG. 11: Shows a close-up of the right part of the headset with the primary control buttons.

FIG. 12: Illustrates a side perspective view of the headset.
12.1: AI system.
12.2: Battery rechargeable by solar and electric power.

FIG. 13: Displays a close-up of the rear view of the headset.
13.1: Solar panels.

FIG. 14: Shows a close-up of the side view of the headset.
14.1: Sensors for measuring temperature and heart rate.

FIG. 15: Illustrates a close-up of the right part of the headset.
15.1: Extendable inner earpiece (right).

FIG. 16: Top perspective view of the headset.

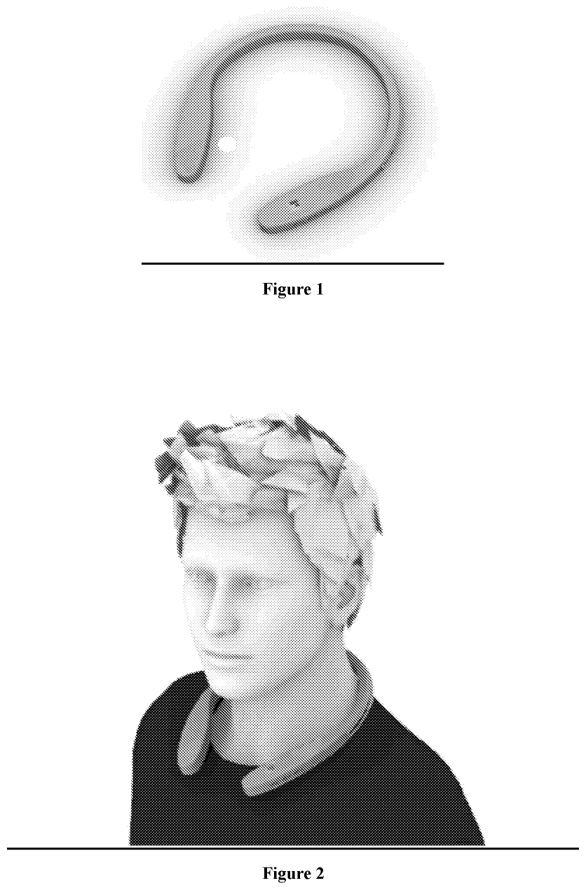

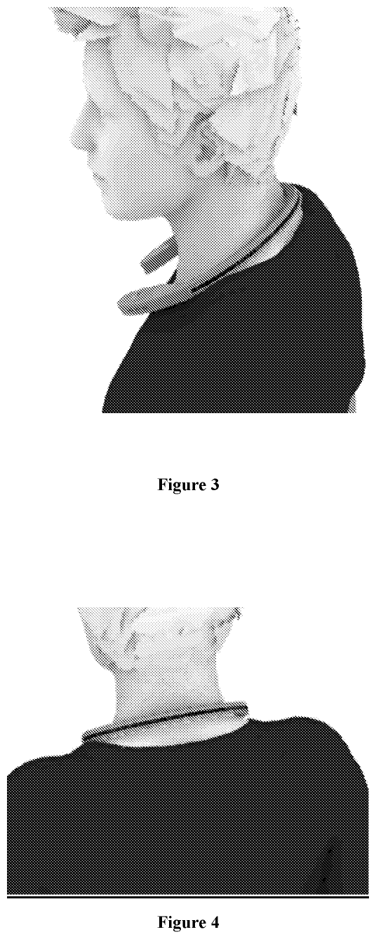

### DETAILED DESCRIPTION

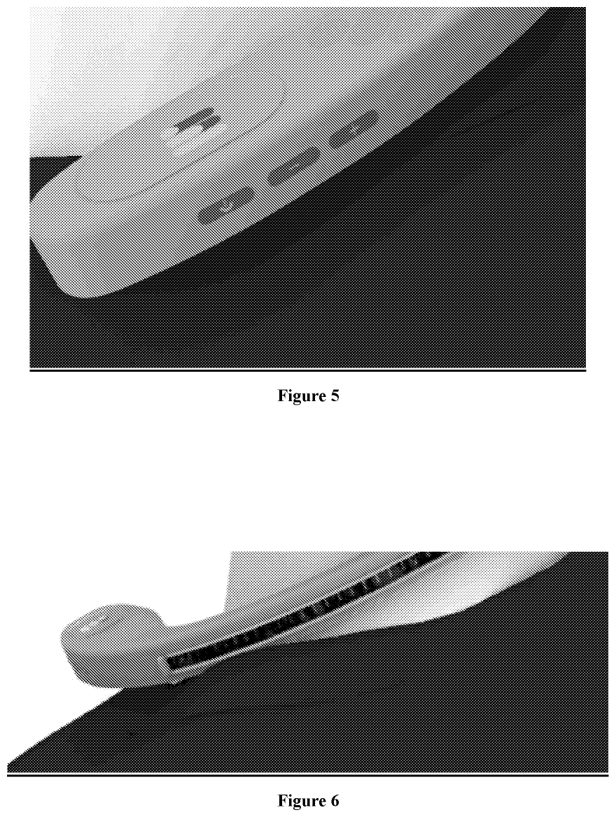

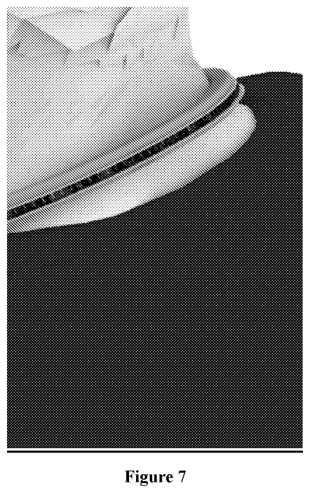

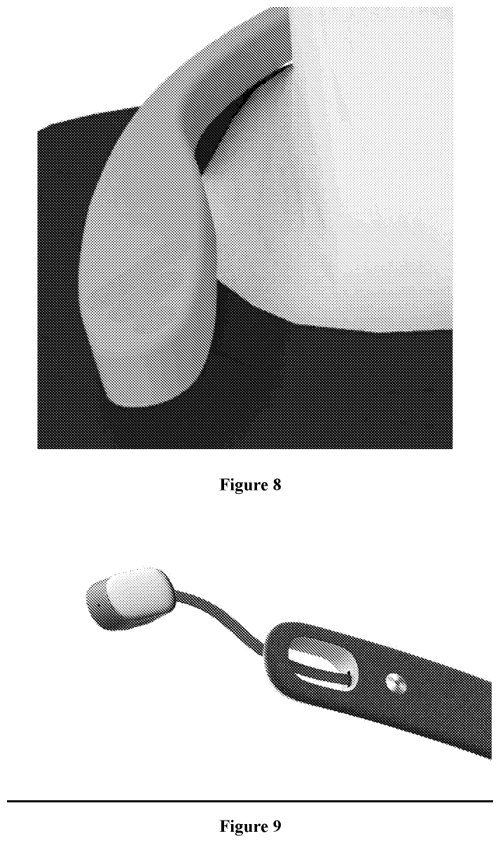

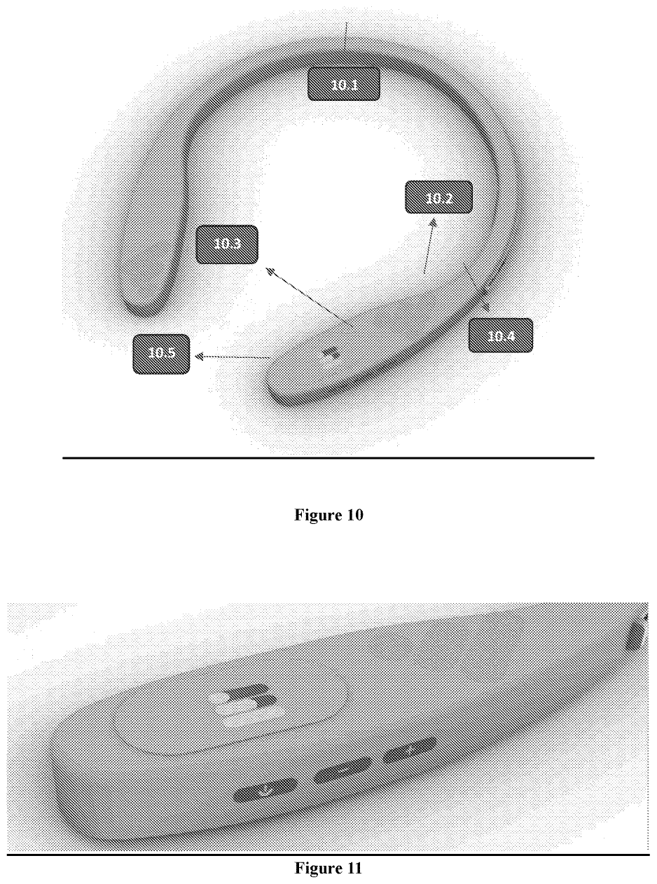

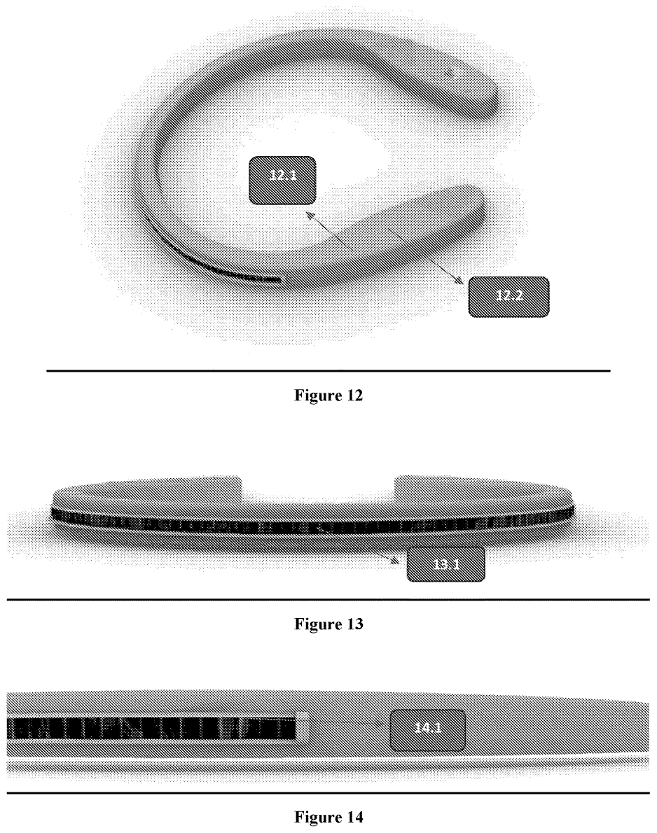

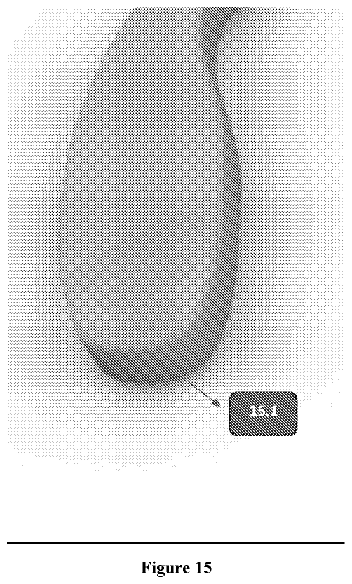

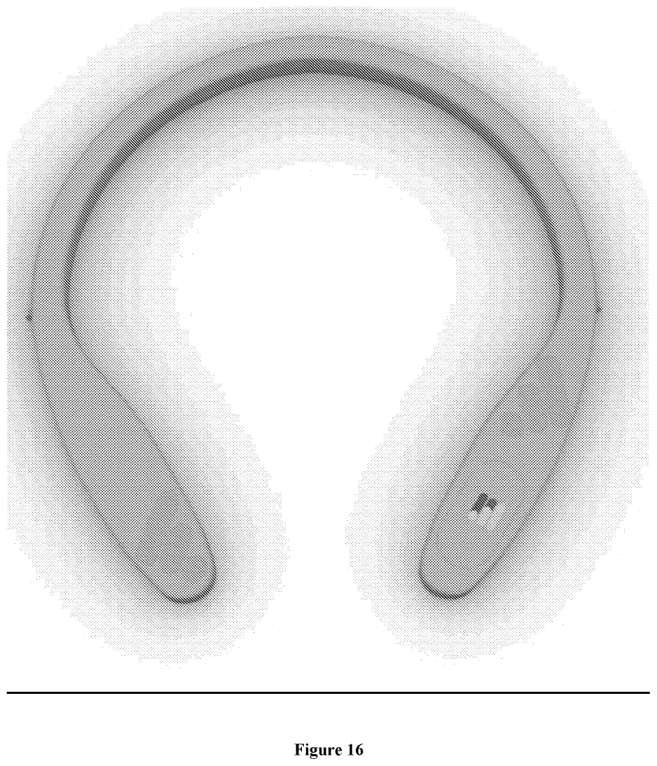

The headset is placed around the neck (FIG. 2). Upon pressing the power button (FIG. 5), the GPS system (FIG. 10.4) is activated, allowing the operating company to monitor and confine all users within their permitted area. The AI system becomes ready to recognize all languages for instant real-time translation (FIG. 12.1). As soon as the microphone is detached from its magnetic holder, it is ready to receive, and process sounds and convert them into the user's language (FIG. 10.3). The sound can be heard either through the inner earpieces (FIGS. 9, 10.5, 15.1) or via the ear-directed speaker (FIG. 10.2) if the inner earpieces are not in use, particularly for emergency alerts. The battery lasts throughout the performance of the rituals and can be recharged using solar panels (FIG. 13.1) or electricity. There are sensors to measure temperature and heart rate (FIG. 14.1) to prevent heatstroke and high blood pressure and to enable early intervention by relevant authorities.

1. The headset is placed around the neck. Upon pressing the power button, the GPS system is activated, allowing the operating company to monitor and confine all users within their permitted area. The AI system becomes ready to recognize all languages for instant real-time translation. As soon as the microphone is detached from its magnetic holder, it is ready to receive, and process sounds and convert them into the user's language. The sound can be heard either through the inner earpieces or via the ear-directed speaker if the inner earpieces are not in use, particularly for emergency alerts. The battery lasts throughout the performance of the rituals and can be recharged using solar panels or electricity. There are sensors to measure temperature and heart rate to prevent heatstroke and high blood pressure and to enable early intervention by relevant authorities.
Key protection elements include:
A lightweight, comfortable, neck-worn headset.
Special rubber material that is anti-sweat and skin-irritation resistant.
Environmentally friendly, recyclable materials.
Extendable and magnetically fixable inner earpieces.
External ear-directed speaker.
Rechargeable battery through solar cells and electricity.
Extendable, magnetically fixable microphone.
Sensors to measure temperature and heart rate.
Solar charging panels.
Instant, real-time translation using AI.
Special and developed AI system.
Reverse translation.
Early intervention.
AI connected via API with a private local network.
GPS system.
A platform for tracking and emergency intervention.
Subsidiary protection elements include:
Headset for people with special needs.
Headset for pilgrims.
Headset for umrah performers.
Headset for tourists.
Headset for businessmen.
Headset for guides.
Samaeati logo.
